(19) United States
(12) Patent Application Publication
(10) Pub. No.: US 2005/0049710 A1
(43) Pub. Date: Mar. 3, 2005

(54) Prosthesis for partial replacement of an articulating surface on bone

(76) Inventors: Shawn W. O'Driscoll, Rochester, MN (US); Bernard F. Morrey, Rochester, MN (US)

Correspondence Address: KOLISCH HARTWELL, P.C., 520 S.W. YAMHILL STREET, SUITE 200, PORTLAND, OR, 97204, US

(21) Appl. No.: 10/927,759

(22) Filed: Aug. 27, 2004

Related U.S. Application Data
(60) Provisional application No. 60/498,807, filed on Aug. 28, 2003.

(51) Int. Cl. A61F 2/38; A61F 2/30

(52) U.S. Cl. 623/20.11; 623/23.44; 623/18.11; 623/20.3

(57) ABSTRACT

Systems, including apparatus, methods, and kits, for replacing a portion of an articulating bone surface with a surface region of a partial prosthesis.

### CROSS-REFERENCE TO PRIORITY APPLICATION

This application is based upon and claims the benefit under 35 U.S.C. § 119(e) of U.S. Provisional Patent Application Ser. No. 60/498,807, filed Aug. 28, 2003, which is incorporated herein by reference in its entirety for all purposes.

### BACKGROUND OF THE INVENTION

Injury to a movable joint may be difficult to repair. In particular, the injury may damage one or more of the articulating surfaces that slide on one another to define movement of opposing bones at the joint. If the shape and position of the damaged articulating surface are not reconstructed accurately, the joint may not function properly. Accordingly, movement of the opposing bones may be limited, unnatural, and/or painful after inaccurate repair of the damaged articulating surface.

A common surgical alternative for reconstruction of an injured joint involves insertion of a prosthesis. The prosthesis may be configured to replace all of an articulating surface on one or both sides of the joint. Accordingly, an entire end of the bone may be resected and the prosthesis affixed to a remaining portion of the bone as a total replacement prosthesis (“a total prosthesis”) for the end of the bone.

Despite its widespread use, the total prosthesis has a number of disadvantages. For example, the total prosthesis may be difficult to position accurately because native bone structures at the end of the bone are removed before the prosthesis is positioned. Accordingly, the total prosthesis may have its artificial articulating surface positioned too close or too far from the opposing bone and/or out of alignment with the opposing bone, among others. In addition, installation of the total prosthesis may replace all native bone structures present at the end of the bone, including regions of undamaged bone that could have been saved. As a result, native bone structures cannot be used for anatomic joint alignment.

The elbow is a common site of joint injury and prosthesis installation. Trauma to the elbow may disrupt articulation of the distal humerus with both of its partners, the proximal ulna and the radial head of the radius. In particular, elbow trauma may dislocate the ulna posteriorly from the humerus, as a result of a fracture of the coronoid process of the ulna. Such trauma also may fracture the radial head to disrupt its proper articulation with the ulna and/or distal humerus. This combination of injuries has been referred to as the “terrible triad” due to its propensity for recurrent dislocation, chronic instability, and poor functional results.

In some cases, terrible triad or other elbow injuries may produce a fracture of the radial head that cannot be reconstructed surgically. To treat these injuries, a total prosthesis for the radial head may be implanted after resection of any unfractured regions of the radial head. However, this resection may remove native bone unnecessarily, destroying natural landmarks for matching the height of the total prosthesis to native bone of the radial head. Even small deviations in the height of the radial head can severely affect elbow function.

Terrible triad or other elbow injuries also may produce coronoid fractures, which may need to be repaired to prevent recurrent dislocation of the elbow. Internal fixation, such as with fasteners (wires, screws, pins, etc.) and/or bone plates, may be used to fix a fractured coronoid fragment to the proximal ulna. However, many coronoid fractures may be difficult to fix due to the shape and size of the resulting coronoid fragments.

### SUMMARY

The present teachings provide systems, including apparatus, methods, and kits, for replacing a portion of an articulating bone surface with a surface region of a partial prosthesis.

### BRIEF DESCRIPTION OF THE DRAWINGS

FIG. 1 is a schematic view of a movable joint defined by a pair of opposing skeletal members, in accordance with aspects of the present teachings.

FIG. 2 is an exploded view of the movable joint of FIG. 1 after resection of an end portion of one of the skeletal members and selection of a partial prosthesis for replacement of the resected end portion, in accordance with aspects of the present teachings.

FIG. 3 is a view of the movable joint and prosthesis of FIG. 2 after the prosthesis has been attached to the one skeletal member in place of the resected end portion to form a hybrid articulating surface, in accordance with aspects of the present teachings.

FIG. 4 is an anterior view of the bones of a left elbow after partial replacement of the coronoid process and the radial head of the elbow with corresponding partial prostheses, in accordance with aspects of the present teachings.

FIG. 5 is a view of the partial radial head prosthesis of FIG. 4 in isolation from the bones of the left elbow.

FIG. 6 is a side elevation view of the partial radial head prosthesis of FIG. 5, viewed generally along line 6-6 of FIG. 5.

FIG. 7 is another side elevation view of the partial radial head prosthesis of FIG. 5, viewed generally along line 7-7 of FIG. 5 and from an opposing side relative to FIG. 6.

FIG. 8 is a fragmentary, anterior sectional view of selected portions of FIG. 4, particularly the radial head and its attached partial radial head prosthesis.

FIG. 9 is a view of the coronoid prosthesis of FIG. 4 in isolation from the bones of the left elbow.

FIG. 10 is a view of the coronoid prosthesis of FIG. 9, generally from an opposing side of the coronoid prosthesis.

FIG. 11 is a side elevation view of the coronoid prosthesis of FIG. 9, taken generally along line 11-11 of FIG. 9.

FIG. 12 is another side elevation view of the coronoid prosthesis of FIG. 9, taken generally along line 12-12 of FIG. 9 and with interior recesses and holes shown in dashed outline.

FIG. 13 is a sectional view of selected portions of FIG. 4, particularly the proximal ulna and coronoid prosthesis of FIG. 4, taken generally along line 13-13 of FIG. 4.

FIG. 14 is a fragmentary sectional view of the proximal ulna and coronoid prosthesis of FIG. 4, taken generally along line 14-14 of FIG. 4.

FIG. 15 is a fragmentary sectional view of the proximal ulna and coronoid prosthesis of FIG. 4, taken generally along line 15-15 of FIG. 4.

FIG. 16 is a view of an alternative coronoid prosthesis that may be used in place of the coronoid prosthesis of FIG. 4, in accordance with aspects of the present teachings.

FIG. 17 is a side elevation view of the coronoid prosthesis of FIG. 16, taken generally along line 17-17 of FIG. 16.

FIG. 18 is a fragmentary, sectional lateral view of a proximal ulna carrying the coronoid prosthesis of FIGS. 16 and 17 and viewed generally as in FIGS. 13 and 14, in accordance with aspects of the present teachings.

### DETAILED DESCRIPTION

The present teachings provide systems, including apparatus, methods, and kits, for replacing a portion of an articulating bone surface with a surface region of a partial prosthesis. Partial replacement may replace one of two adjoining regions within the articulating surface to form a hybrid articulating surface that includes native and prosthetic surface regions. Alternatively, or in addition, partial replacement may form a hybrid articulating surface by replacing one of two natural articulation regions that alternately articulate with a common site on an opposing skeletal member. The apparatus may include partial prostheses that provide implanted surface regions corresponding to a portion of a natural articulating surface. The partial prostheses may be configured to abut a cut surface of a bone. The partial prostheses may be configured to be affixed to the bone using fasteners, adhesives, and/or projections, among other. Partial prostheses, as described herein, may provide a number of advantages over total prostheses, including increased preservation of natural articulating surfaces, improved positioning of artificial surface regions for articulation, and/or better attachment to native bone.

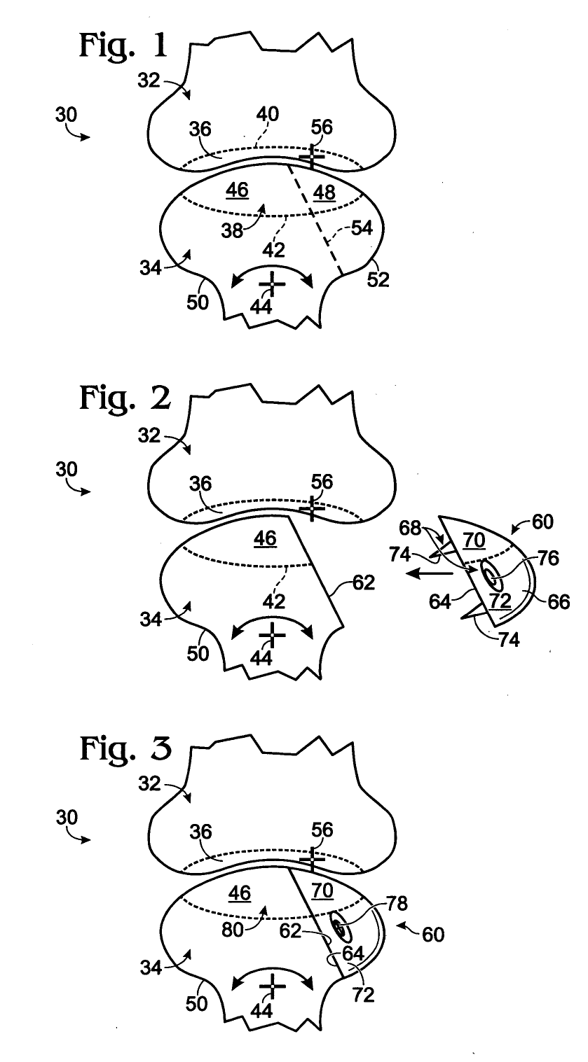

FIG. 1 shows a schematic view of a movable joint 30 defined by the ends of a pair of opposing skeletal members or bones, such as proximal bone 32 and distal bone 34. Bones 32, 34 may contact one another through articulating surfaces, such as proximal articulating surface 36 and distal articulating surface 38, respectively, defined within articulation perimeters 40, 42.

Movement of distal articulating surface 38 along opposing proximal surface 36 may guide and/or restrict movement of bones 32, 34 relative to one another. For example, distal bone 34 may pivot about an axis 44 by sliding distal articulating surface 38 relative to a complementarily shaped region of proximal surface 36. In some joints, distal bone 34 may pivot about one or more additional axes, for example, torsional movement around its long axis, pivotal movement about an axis orthogonal to axis 44, and/or or translational movement along a more planar opposing articulating surface.

Distal bone 34 may include a plurality of adjoining articulating surface regions within distal articulating surface 38. For example, first and second adjoining surface regions 46, 48 may be defined by a major bone portion 50 and a minor bone portion 52, respectively, on opposing sides of a resection boundary 54 at which the surface regions meet. Surface regions 46, 48 may be placed alternately in apposition with a common site or point 56 within articulation region 36 of proximal bone 32 by relative movement of bones 32, 34, such as by rotation of distal bone 34 about axis 44.

FIG. 2 shows an exploded view of movable joint 30 after resection of minor bone portion 52 and selection of a partial prosthesis 60 for replacement of minor bone portion 52 (see FIG. 1). Distal bone 34 has been cut along resection boundary 54 (see FIG. 1) to produce cut surface 62 on the distal bone and to remove surface region 48 of articulating surface 38. In some embodiments, a portion of proximal bone 32 may be resected, and the prosthesis may be configured for attachment to the proximal bone. Furthermore, each of two or more opposing bones may be resected and fitted with a partial prosthesis.

Partial prosthesis 60 may include an inner surface 64, an outer surface 66, and attachment features 68, among others. The inner surface 64 may be disposed in apposition to cut surface 62. Accordingly, the inner surface may be generally complementary to cut surface 62, or a portion thereof, and may be internal to a bone-prosthesis hybrid. The outer surface 66 may be on the exterior of the bone-prosthesis hybrid. The outer surface may define an implanted surface region or replacement surface region 70 configured to articulate movably with opposing articulating surface 36 of proximal bone 32. The outer surface also may define additional external (nonarticulating) surface region 72, which may perform structural and/or functional roles, as described in more detail below. Attachment features may include one or more projections extending from inner surface 64, such as prongs 74, a hole 76 for receiving a fastener 78 (see FIG. 3), and/or the like.

FIG. 3 shows movable joint 30 with partial prosthesis 60 attached to major portion 50 of distal bone 34. Cut surface 62 may be disposed in apposition with inner surface 64. A hybrid articulation surface 80 may be defined by a native bone surface and a prosthetic surface, particularly native surface region 46 and artificial surface region 70. Pivotal movement of bone 34 and prosthesis 60 about axis 44 may place native and implanted surface regions 46, 70, respectively, in alternate contact with common site 56 on opposing bone 32.

Further aspects of the present teachings are described in the following sections, including (I) movable joints and articulating surfaces, (II) partial prostheses, and (III) examples.

I. Movable Joints and Articulating Surfaces

Partial prostheses may replace portions of a skeleton at any suitable movable joints. Movable joints, as used herein, may be any skeletal junctions at which two, three, or more skeletal members meet in movable contact. Movable joints may include a joint of an elbow, an ankle, a knee, a hip, a wrist, a shoulder, an intervertebral junction, a hand, a foot, a finger, or a toe, among others. Accordingly, a partial prosthesis may replace part of a proximal or distal end of the humerus, ulna, radius, femur, fibula, tibia, vertebra, etc.

Each skeletal member at a movable joint may be a structural support formed of natural bone and/or implanted support material. Natural bone may be any bone produced in the body, including native bone or transplanted bone, among others. Implanted support material may be any man-made structural support connected to the skeleton in place of, or in addition to, natural bone. Exemplary implanted support material may include total or partial prostheses, medical implants, bone plates, and/or the like. Accordingly, partial prostheses, as described herein, may articulate with natural bone, with implanted support material, or a combination thereof. In some examples, a partial prosthesis secured to one bone may articulate with another partial prosthesis secured to an adjacent bone.

A partial prosthesis may replace a region of a natural articulating surface with an implanted surface to form a hybrid articulation surface. An articulating surface, as used herein, may be a substantially continuous contact surface, on an end segment of a bone, that slides relative to and along an opposing natural and/or artificial (or implanted) surface of an opposing skeletal member. A substantially continuous contact surface is a surface that is not divided into a plurality of spaced articulation regions by a nonarticulating (noncontacting) spacer region. A nonarticulating spacer region is disposed, for example, between lateral and medial articulating surfaces of the tibia. Accordingly, the end segment of a bone may have a plurality of spaced articulating surfaces that contact different opposing bones or nonadjoining regions of the same opposing bone. Each articulating surface may be composed of a plurality of surface regions. The surface regions may be contiguous (adjoining), that is, they may abut one another. Alternatively, or in addition, the surface regions may articulate with a common articulation site or point on an opposing skeletal member, as described above. Each substantially continuous articulating surface has a perimeter, and the prosthesis may replace a region of the articulating surface that extends to the perimeter (and also may replace non-articulating surface regions outside the perimeter).

A natural articulating surface may be defined by any suitable material. For example, the natural articulating surface may be defined by one or more layers of connective tissue, such as cartilage, disposed on osseous (subchondral) bone material.

II. Partial Prostheses

Partial prostheses are provided for connection to end segments of bones. A partial prosthesis, as used herein, is any prosthesis or implant configured to be connected to bone, to replace less than all of a natural articulating surface with an implanted surface for contact with an opposing skeletal member. The implanted surface (or an articulating portion thereof) is configured to substitute for or replace a missing or removed portion of bone. Accordingly, the implanted surface makes movable contact with an opposing skeletal member to form a partial replacement joint by partial hemiarthroplasty. The implanted surface may correspond to a replaced portion of the natural articulating surface, that is, the implanted surface may approximate the surface contours of the replaced portion. The prosthesis also may have a surface region that corresponds to nonarticulating surfaces that were removed from the bone.

A partial prosthesis may be formed as a single piece or component, or as a plurality of connected pieces or components. In some embodiments, the partial prosthesis may include a plurality of partial prostheses connected by a fixed or flexible connector, for example, to provide partial and/or total replacement of a corresponding plurality of articulating surfaces, as on two adjacent bones.

A partial prosthesis may have any suitable composition. Partial prostheses may be formed at least substantially of a biocompatible, non-bone material, such as metal (for example, cobalt chromium alloy, a titanium alloy, stainless steel, etc.), ceramic (such as alumina, hydroxyapatite, etc.), plastic (such as ultra-high molecular weight polyethylene or the like) and/or a combination thereof, among others. In some embodiments, partial prostheses may have a body formed of a cobalt chromium alloy or a porous form of titanium or titanium alloy, such as a mesh.

Inner and/or outer surfaces of the prostheses may have compositions and/or textures that are different than one another and/or different than the body of the prosthesis, for example, as a result of a surface treatment or addition. In some embodiments, the outer surface, particularly an artificial articulating region of the outer surface, may be formed by addition of a layer of a biological or synthetic material to the body. Exemplary biological or synthetic materials may include natural or synthetic cartilage, a gel (such as a polyvinylalcohol hydrogel), a layer(s) of cells or tissue, and/or a polymer, among others. In some embodiments, the inner surface of the body of a prosthesis may be configured to promote bone adhesion, such as by including pores and/or a relatively rough texture (that is, rougher than the outer surface). In some examples, the inner surface may be defined by a porous matrix. The porous matrix may be formed with bone chips, bone powder, or with a porous and/or mesh form of titanium or a titanium alloy, among others. In some embodiments a porous material may fill a pocket or recessed region formed by the inner surface of the prosthesis and/or may be configured as a coating. In exemplary embodiments, a titanium plasma may be applied to an inner surface of the prosthesis, such as by spraying, to form a relatively rough surface to promote bone adhesion.

The inner surface of a partial prosthesis may have any suitable shape. The inner surface may be configured to complement the cut surface of a bone. Accordingly, the inner surface may be substantially planar, to match a planar cut surface, or may define a plurality of transverse and/or approximately orthogonal planar surfaces, a convex conical surface, a cylindrical surface, a convex spherical surface (a portion of a sphere), and/or the like. The inner surface also may include one or more projections to facilitate positioning and/or connection of the prosthesis to bone. For example, the projections may be generally cylindrical, tapered, and/or conical, among others. The projections may be configured as posts to be received in holes formed artificially in the bone, such as by drilling, or may be configured to be received in a natural cavity in the bone, such as the medullary canal. Alternatively, or in addition, the projections may be configured to taper to a sharp end, such as to form prongs. Prongs may be used, for example, to create holes as the prosthesis is pressed against and/or into the bone. In any case, the projections may limit movement of the prosthesis relative to bone, for example, provisionally as other fastener mechanisms, such as screws, are being implemented, and/or more permanently, in conjunction with other fastener mechanisms.

The inner surface may be even and/or smooth or may include any suitable surface irregularities. In some embodiments, the inner surface may include an elevated perimeter that creates a central recess. The elevated perimeter may be formed by a lip or ridge that extends partially or completely around the perimeter of the inner surface. The central recess may be left unfilled or may be partially or completely filled or coated with a porous material, as described above, to facilitate, for example, bone growth onto the inner surface of the prosthesis (and/or into the prosthesis).

The outer surface may define an implanted contact (articulating) region of any suitable shape. The implanted contact region may be configured to correspond to or approximate the anatomical contour of the articulation region. The outer surface may provide an implanted or artificial articulation region that defines a similar set of contact points with the opposing skeletal member as the natural articulation region that was replaced. However, the artificial articulation region of the prosthesis may deviate somewhat from the anatomy of the natural region that was replaced, for example, to facilitate positioning of the artificial articulation region in a wider range of anatomies in the population. In some embodiments, the artificial articulation region of the prosthesis may be a plurality of spaced contact regions for articulation with different bones, with spaced regions on the same bone, or with a common region on the same bone. Accordingly, the prosthesis may include spaced articulation regions separated by nonarticulating surfaces that do not correspond to the natural articulation region. For example, the natural articulating surface may be generally smooth or even and the artificial surface may be uneven, that is, including bumps, ridges, depressions, or grooves, for example, to alter distribution of the load on the prosthesis and adjoining natural bone.

The outer surface of the prosthesis also may define a nonarticulating surface region that is configured not to contact bone. The nonarticulating region may have a shape similar to the anatomy of corresponding natural bone surfaces or may be distinct from such anatomy. The nonarticulating region may be configured to interface with tendons or other tissues. Accordingly, the nonarticulating region may be smooth and free of sharp edges. In some embodiments, the nonarticulating region may be textured.

A partial prosthesis may include one or more holes for receiving fasteners. The holes may be threaded or nonthreaded. The holes may be configured to receive screws, wires, or other fasteners, either with or without threaded engagement. In some embodiments, the holes may have a counterbore disposed adjacent the outer surface of the prosthesis and configured, for example, to receive a head of a screw or related fastener. In this case, a proximal portion of the screw's shaft, adjacent the head, may extend through the prosthesis and a distal portion may extend into bone. Alternatively, or in addition, one or more of the holes may be threaded, to engage a proximal or distal portion of the threaded shaft of a screw. Accordingly, such holes may be configured to receive a fastener inserted first through bone and then received in the prosthesis.

A partial prosthesis may include any other suitable fastening mechanisms. Exemplary fastening mechanisms may include a hook, a clip, a loop, or a belt, among others, to engage or extend around bone. In some embodiments, the partial prosthesis may be configured to be attached to bone using an adhesive, such as bone cement, alone or in conjunction with other fastener mechanisms.

Partial prostheses may differ in size and/or handedness, among others. Each partial prosthesis may be configured for use on both the left and right sides of the body, or may be configured specifically for only the left or right side, such as a prosthesis for partial replacement of the coronoid process of the left ulna. In addition, each partial prosthesis may have a size based on a relative bone size, for example, small, medium, or large, for people with small, medium, or large bones (or bone features), respectively. Alternatively, or in addition, each partial prosthesis may have a size according to the amount or fraction of articulating surface to be replaced. For example, different sizes of coronoid prostheses may be configured to replace only a tip of the coronoid process, about one-half of the coronoid process, and a substantial portion or all of the coronoid process, among others.

Partial prostheses may include indicia to identify aspects of the prostheses. Such aspects may include a bone region for which the prosthesis is configured, a left- or a right-handed configuration, size within a set of related prostheses (such as S, M, L, and XL), amount of articulating surface to be replaced, etc. Suitable indicia may include colors, alphanumeric characters, symbols, barcodes, and/or the like.

Partial prostheses may be supplied in a kit. The kit may include a set of prostheses for different bones or articulating surfaces, with different handednesses, different ranges of sizes, different fractions of the articulating surface to be replaced, etc. The kit also may include fasteners for use with the prostheses, bits for drilling holes for the fasteners, cutting devices for removing bone material, and/or instructions, among others.

### III. EXAMPLES

The following examples describe selected aspects and embodiments of the present teachings, including prostheses for partial replacement of a radial head or a coronoid process, both from the elbow. These examples and the various features and aspects thereof are included for illustration and are not intended to define or limit the entire scope of the present teachings.

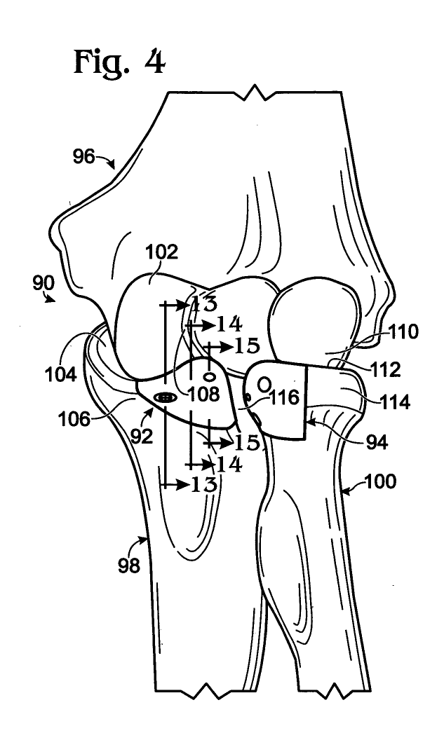

FIG. 4 shows an anterior view of the bones of the left elbow 90, following installation of an exemplary coronoid prosthesis 92 and an exemplary partial radial head prosthesis 94. Alternatively, either of these prostheses may be installed alone in the elbow. Left elbow 90 includes a distal region of the humerus 96 and proximal regions of the ulna 98 and radius 100. These bones appose and articulate with one another. In particular, the trochlea 102 of the humerus articulates with the trochlear notch 104 defined by a proximal aspect of ulna 98. The coronoid process 106, which has been partially removed and replaced with coronoid prosthesis 92, defines an anterior portion of the trochlear notch and rides in a concave depression or trochlear groove 108 of trochlea 102. Furthermore, the capitulum 110 of the distal humerus articulates with a concave depression 112 defined by the radial head 114 of the radius. A portion of the radial head has been removed in the present illustration and replaced with partial radial head prosthesis 94. The side of the radial head also articulates with the radial notch 116, defined by a lateral surface of the ulna, as concave depression 112 of the radial head articulates with capitulum 110. In some embodiments, the coronoid prosthesis may replace at least a portion (or all) of the radial notch with implanted material. Accordingly, with various configurations of the coronoid prosthesis, this prosthesis may not articulate with the radius or may articulate with the natural radial head, a total radial head prosthesis, and/or a partial radial head prosthesis, among others.

The structure and installation of partial radial head prosthesis 94 and two alternative coronoid prostheses 92, 230 are described below in more detail.

### Example 1

### Prosthesis for Partial Radial Head Replacement

This example describes the structure and installation of exemplary partial radial head prosthesis 94; see FIGS. 4-8.

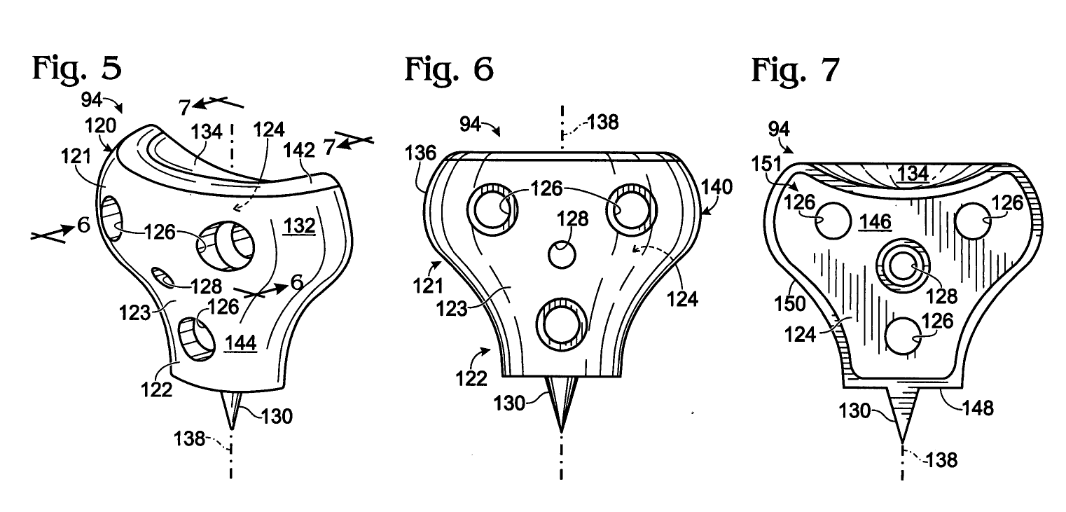

FIGS. 5-7 show partial radial head prosthesis 94 of FIG. 4 in isolation from the bones of left elbow 90. Partial radial head prosthesis 94 may include a body 120 having a proximal head 121 and a distal neck 122. The head and neck may define an outer surface 123 and an inner surface 124. The body also may define a plurality of openings or holes 126, 128 for receiving fasteners. A prong 130 may be connected to the body, for example, extending from a distal region of inner surface 124. In alternative embodiments, the prong may be a stem configured to extend into a natural bone cavity, such as the medullary canal.

Outer surface 123 may include a plurality of spaced surface regions 132, 134 for articulation with the ulna and the humerus, respectively. Lateral or side surface region 132 may be configured to articulate with radial notch 116 of the ulna. Lateral region 132 may define a cylindrical shape, as is found in an articulating lateral region of the native radial head. Alternatively, lateral region 132 may be more spherically shaped, to define an arcuate profile 136 when viewed orthogonal to axis 138 of the prosthesis (see FIG. 6). Accordingly, lateral region 132 may have a greatest diameter (measured orthogonal to axis 138) at an axial position that is substantially centered within the side region, shown at 140. Top or end surface region 134 may be disposed on a proximal surface of the prosthesis. Top surface region 134 may be configured to articulate with capitulum 110 in cooperation with radial head surface region 112 (see FIG. 4). Accordingly, top surface region 134 may define a portion of an axial depression on the radius, when installed, and may have a concave, spherical shape.

Outer surface 123 also may include nonarticulating surface regions 142, 144. Rim surface region 142 may form a spacer between articulating regions 132, 134. Neck surface region 144 may adjoin articulating surface region 132 and may be included in a tapered portion of neck 122.

Inner surface 124 may include inner surface regions 146, 148 (see FIG. 7). Axial inner region 146 may be configured to extend at least substantially parallel to axis 138 of the prosthesis and to the long axis of the radius. Axial inner region 146 may have a lip 150 that extends around the perimeter of the inner region, so that the majority of inner region 146 is recessed. In some embodiments, the recessed portion 151 of the inner region 146 may be filled or coated with a porous material, such as a titanium plasma alloy, as described above, so that inner region 146 is substantially planar. Transverse inner region 148 may be configured to extend transversely (obliquely or orthogonally) to axis 138 of the prosthesis and to the long axis of the radius.

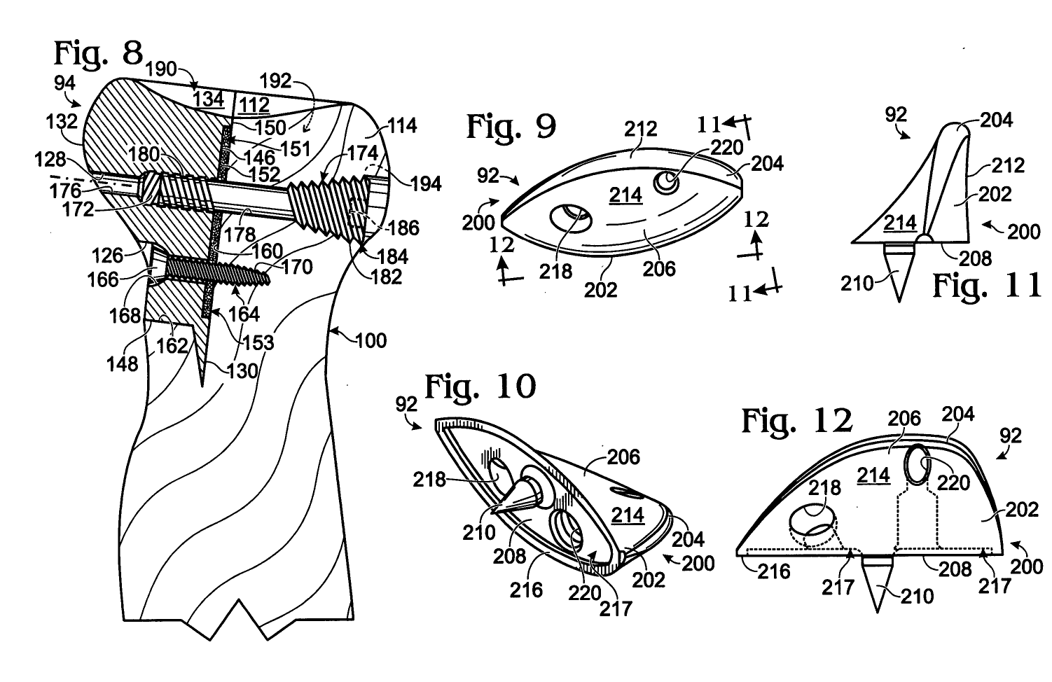

FIG. 8 shows an anterior sectional view of radius 100, particularly radial head 114, and partial radial head prosthesis 94 of FIG. 4. To simplify the presentation in this and other figures, the medullary canal is not shown. In the present illustration, recessed portion 151 of prosthesis 94 has been filled with a porous material 152 to define a new composite inner surface region 153. Radius 100 may be prepared for attachment of prosthesis 94 by removing a suitable segment of the radial head. The radial head may be cut axially, to create axial sectional surface 160, and cut generally transverse to the axis of the radius to create transverse sectional surface 162. Prosthesis 94 may be positioned against cut surfaces 160, 162, so that composite inner surface region 153 abuts axial sectional surface 160 and transverse surface 162 abuts transverse inner region 148. This placement of the prosthesis against the proximal radius may be facilitated and provisionally stabilized by prong 130 extending axially into the radius.

Prosthesis 94 may be affixed to the radius using bone screws received in holes 126, 128. Hole 126 may be nonthreaded (or threaded) and may be configured to receive bone screw 164 so that head 166 of the bone screw engages the prosthesis in a counterbore 168 of hole 126, and a distal region of shaft 170 of the bone screw engages the radius. Hole 128 may be threaded, shown at 172 and may be configured to receive a bone screw 174 from an opposing direction.

A bore in bone may be created for receiving bone screw 174 from the opposing direction in two steps. In a first step, hole 128 of the prosthesis may be used as an entry point and as a guide for drilling a guide channel of smaller diameter extending from the prosthesis to an opposing external surface of the radial head. For example, hole 128 may include a nonthreaded bore 176 disposed adjacent a threaded region of the hole. In some embodiments, a guide wire may be placed through the radius from bore 176, along a path extending linearly from this bore. In a second step, the guide channel (or guide wire) may serve as a guide for drilling a bore 178 of larger diameter, from the radial head toward the prosthesis. For example, a cannulated drill bit may receive the guide wire in an axial bore of the drill bit and travel along the guide wire during drilling. Screw 174 then may be inserted into bore 178 and rotated into threaded engagement with threads 172 of the prosthesis. Accordingly, screw 174 may include machine threads 180 of a constant diameter and pitch in its distal end region. A head of screw 174 may includes tapered threads 182 of constant or varying pitch to define a head 184 of screw 174. Head 184 may include a tool engagement structure, such as an axial recess 186, a slot(s), etc.

Partial radial head prosthesis 94 may form a plurality of spaced, hybrid articulation regions. Axial hybrid region 190 for articulation with the capitulum may be defined by radial head surface region 112 and prosthesis surface region 134. Lateral hybrid surface region 192 may be defined by a side surface region 194 of the radial head and lateral surface region 132 of the prosthesis.

### Example 2

### Prosthesis for the Coronoid Process

This example describes the structure and installation of exemplary coronoid prosthesis 92; see FIGS. 4 and 9-15.

FIGS. 9-12 show various views of coronoid prosthesis 92 in isolation from the bones of the left elbow. Coronoid prosthesis 92 may include a body 200 having a base 202 and a ridge 204 extending between opposing ends of the base. Body 200 may define an outer surface 206 and an inner surface 208. One or more prongs 210 or other projections may be connected to body 200 adjacent the inner surface, such that the projections may extend into the ulna when the prosthesis is installed.

Outer surface 206 may include distinct surface regions. Outer surface 206 may include an articulation or contact surface region 212 (see FIG. 9) for contact with trochlea 102 (see FIG. 4). Accordingly, articulation region 212 may be configured to approximate the articulation contour of a distal portion of the coronoid. Outer surface 206 also may include nonarticulating surface region 214 that generally opposes articulation region 212 on an opposing side of ridge 204.

Inner surface 208 (see FIG. 10) may be configured to face a cut surface of the ulna. Inner surface 208 may include a lip 216 that extends partially or completely around the perimeter of the inner surface. The lip may create a recessed region 217 central to the lip. The recessed region may be filled or coated with a porous material or left unfilled or uncoated.

Body 200 also may define one or a plurality of openings or holes 218, 220 (see FIG. 12) for receiving fasteners such as bone screws. Each hole may be threaded or nonthreaded, and may be configured for receiving a bone screw from outer surface 206 and/or from inner surface 208, as described below.

FIGS. 13-15 show sectional views of the proximal ulna 98 and coronoid prosthesis 92, generally according to sections indicated in FIG. 4. In these views, recessed region 217 of prosthesis 92 (see FIGS. 10 and 12) has been filled with a porous material and is not visible, to simplify the presentation. To provide an attachment surface for the coronoid prosthesis, ulna 98 may be cut to create cut surface 222. The cut may be substantially planar and may extend at any suitable angle to the axis of the ulna, based on the configuration of the coronoid prosthesis and the amount of the coronoid process that is removed. Attachment of coronoid prosthesis 92 may form a hybrid articulation region 223 using trochlear notch 104 and outer surface region 212 of the prosthesis.

FIG. 13 shows bone screw 224 extending through hole 218 and attaching prosthesis 92 to the ulna. Bone screw 224 may engage prosthesis 92 with a head of the bone screw and may engage bone with distally disposed threads of the bone screw, as described above for bone screw 164 in relation to FIG. 8. In some examples, bone screw 224 also may engage the coronoid prosthesis threadably.

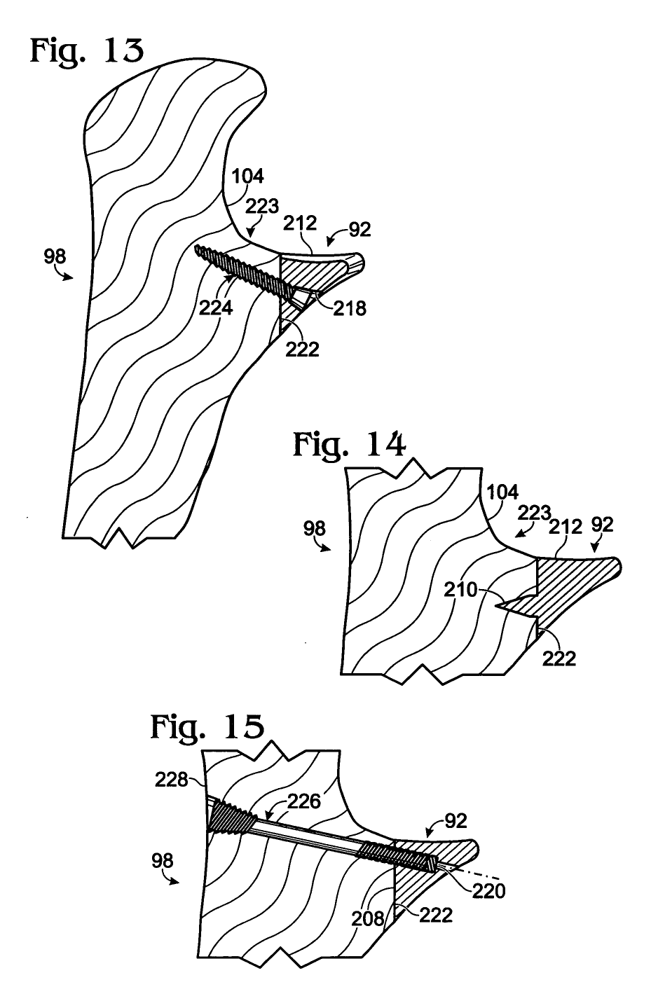

FIG. 14 shows prong 210 extending below cut surface 222. Prong 210 may form a recess in bone as the prong is urged into bone. Alternatively, or in addition, prong 210 may be received in a preformed and/or natural hole in the bone.

FIG. 15 shows bone screw 226 extending into hole 220 of prosthesis 92 from inner surface 208. Screw 226 may be installed and engaged with bone and the prosthesis as described above for screw 174 in relation to FIG. 8. Screw 226 may extend at any suitable angle relative to the ulna axis, for example, orthogonal to the axis or oblique to the axis. In addition, screw 226 may extend with its head adjacent an opposing surface 228 of bone and its distal shaft in threaded engagement with the prosthesis.

### Example 3

### Alternative Prosthesis for the Coronoid Process

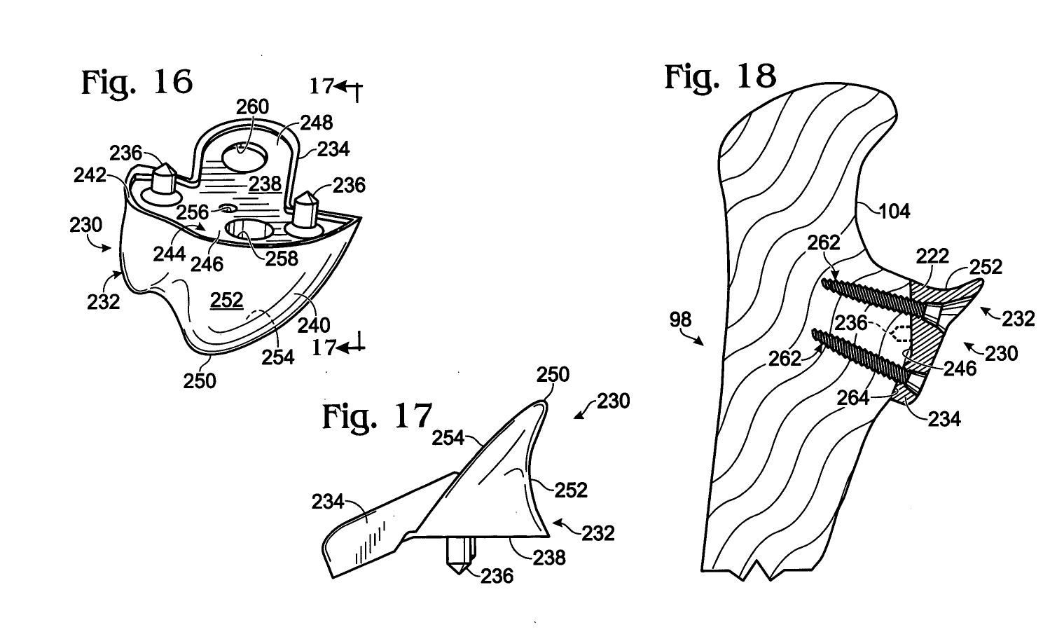

This example describes the structure and installation of an alternative exemplary coronoid prosthesis; see FIGS. 16-18.

FIGS. 16 and 17 are views of an alternative coronoid prosthesis 230 that may be used in place of coronoid prosthesis 92. Coronoid prosthesis 230 may include a body 232, a tab 234 connected to the body, and one or more projections, such as prongs 236 extending from the body.

The body, the tab, and the projections may define an inner surface 238 and an outer surface 240. Inner surface 238 may include a lip 242 that extends partially or completely around a perimeter of the inner surface. Lip 242 may create a recessed region 244 central to the lip. The recessed region may be unfilled or filled (or coated) before installation of the prosthesis, as described above. In alternative embodiments, the lip may not be included in the prosthesis. Inner surface 238 may include a body inner surface 246 and a tab inner surface 248. These inner surfaces may be parallel or nonparallel. In the present illustration, the surfaces extend obliquely to one another. One or more projections may extend from either or both of the body inner surface and the tab inner surface. Outer surface 240 may define a ridge 250 and surface regions 252, 254 on opposing sides of the ridge. Some or all of surface region 252 may be configured to articulate with the trochlea of the humerus. Opposing surface region 254 may be substantially or completely nonarticulating with an opposing skeletal member.

Body 232 and/or tab 234 may define one or a plurality of openings or holes for receiving fasteners. For example, body 232 may define holes 256, 258, which are configured for receiving a wire and a screw, respectively. In addition, tab 234 may define a hole(s) 260 for receiving a bone screw and/or a wire. The holes may be threaded or nonthreaded.

FIG. 18 shows a sectional lateral view of ulna 98 carrying coronoid prosthesis 230. This view is generally taken as in FIGS. 13 and 14. Bone screws 262 may extend through the prosthesis and into bone, so that the heads of the bone screws engage the prosthesis. Body inner surface 246 may appose cut surface 222 of the bone and tab inner surface 248 may appose uncut external bone surface 264.

### Example 4

### Selected Embodiments

This section describes selected embodiments of the present teachings, presented as a series of indexed paragraphs.

1. A prosthetic device for partial replacement of an articulating surface of a movable joint, the movable joint including a bone defining the articulating surface and an opposing skeletal member that contacts the articulating surface, the device comprising:
a body configured to be connected to the bone to replace one of two contiguous regions of the articulating surface, the body including a replacement surface so that a hybrid articulation region is formed with the replacement surface and the other contiguous region for movable contact with the opposing skeletal member.

2. The prosthetic device of paragraph 1, wherein the movable joint is an elbow.

3. The prosthetic device of paragraph 1, wherein the articulating surface has a perimeter, and wherein the one contiguous region extends to the perimeter.

4. The prosthetic device of paragraph 2 or 3, wherein the bone is a radius and the articulating surface is defined by a proximal end segment of the radius.

5. The prosthetic device of paragraph 4, wherein the replacement surface is configured to articulate with the ulna adjacent the radius.

6. The prosthetic device of paragraph 4, wherein the replacement surface is configured to articulate with the capitulum of the humerus.

7. The prosthetic device of paragraph 6, wherein the replacement surface further is configured to articulate with the ulna.

8. The prosthetic device of paragraph 2 or 3, wherein the bone is an ulna and the body is configured to replace at least a portion of the coronoid process of the ulna.

9. The prosthetic device of paragraph 1, wherein the movable joint is a knee.

10. The prosthetic device of paragraph 1, wherein the body defines at least one aperture configured to receive fasteners to connect the body to the bone.

11. The prosthetic device of paragraph 10, wherein at least one of the apertures is configured to receive a bone screw that is introduced first through bone and then into the aperture.

12. The prosthetic device of paragraph 11, wherein the at least one of the apertures is at least partially threaded.

13. The prosthetic device of paragraph 11, wherein at least another of the apertures is configured to receive a bone screw that is introduced first through the aperture and then into bone.

14. The prosthetic device of paragraph 13, wherein the at least another of the apertures is at least partially threaded.

15. The prosthetic device of paragraph 11, further comprising at least one projection that extends from the body, the projection being configured to be received in the bone to limit movement of the body.

16. The prosthetic device of paragraph 15, wherein the long axis of at least one of the apertures and the long axis of at least one of the projections are at least substantially perpendicular.

17. The prosthetic device of paragraph 15, wherein the long axis of at least one of the apertures and the long axis of at least one of the projections are at least substantially parallel.

18. The prosthetic device of paragraph 1, further comprising at least one projection that extends from the body, the projection being configured to be received in the bone to limit movement of the body.

19. The prosthetic device of paragraph 18, wherein the at least one projection includes a distal end region that is pointed.

20. The prosthetic device of paragraph 19, wherein the at least one projection is a prong configured to be pressed into the bone.

21. The prosthetic device of paragraph 1, wherein the body defines an inner surface configured to face bone, the body including a lip that borders a recessed portion of the inner surface.

22. The prosthetic device of paragraph 21, wherein the recessed portion is at least partially coated with a porous material.

23. The prosthetic device of paragraph 1, wherein the body defines an inner surface configured to appose bone, the inner surface being generally planar.

24. The prosthetic device of paragraph 1, wherein the body defines a pair of inner surfaces configured to face bone, the two inner surfaces being at least approximately perpendicular to one another.

25. A prosthetic device for partial replacement of an articulating surface on a bone that articulates with an opposing skeletal member in a movable joint, comprising:
a body configured to be connected to the bone to replace one of two surface regions of the articulating surface for movable contact with the opposing skeletal member, the surface regions alternately contacting a surface site on the opposing skeletal member as the bone moves relative to the opposing skeletal member.

26. The prosthetic device of paragraph 25, wherein the body includes a replacement surface corresponding to the one surface region so that a hybrid articulation region is formed with the replacement surface and the other surface region for contact with the opposing skeletal member.

27. The prosthetic device of paragraph 25, wherein the articulating surface has a perimeter, and wherein the one surface region extends to the perimeter.

28. The prosthetic device of paragraph 25, wherein the articulating surface is provided by the radial head of a radius.

29. The prosthetic device of paragraph 25, wherein the articulating surface is provided by a coronoid process of an ulna.

30. The prosthetic device of paragraph 25, wherein the body is configured to abut a cut surface of bone that is at least substantially planar.

31. A prosthetic device for partial replacement of a native articulating surface on an end segment of a bone, the native articulating surface being substantially continuous and having a perimeter, the device comprising: a body configured to be connected to the bone and including a replacement surface for a missing portion of the native articulating surface so that a hybrid articulating surface is formed for movable contact with an opposing skeletal member, wherein the missing portion includes part of the perimeter.

32. A method of repairing an articulating surface of a bone, the articulating surface contacting an opposing skeletal member in a movable joint, comprising: A) cutting the bone to remove a natural region of the articulating surface and create a cut surface; and B) attaching a prosthesis to bone adjacent the cut surface so that the natural region of the articulating surface is replaced with an artificial region configured to contact the opposing skeletal member movably.

33. The method of paragraph 32, wherein the step of cutting the bone creates a substantially planar surface, and wherein the step of attaching a prosthesis includes contacting the substantially planar surface with a corresponding inner surface of the prosthesis.

34. The method of paragraph 33, wherein the step of cutting the bone creates a plurality of substantially planar surfaces that are nonparallel, and wherein the step of attaching a prosthesis includes contacting the plurality of substantially planar surfaces with a corresponding plurality of inner surfaces of the prosthesis.

35. The method of paragraph 32, wherein the step of attaching a prosthesis includes fastening the prosthesis to the bone with a bone screw.

36. The method of paragraph 35, wherein the step of fastening the prosthesis to the bone with a bone screw includes introducing the bone screw first through an aperture in the prosthesis and then into the bone.

37. The method of paragraph 35, wherein the step of fastening the prosthesis to the bone with a bone screw includes introducing the bone screw first through the bone and then into an aperture in the prosthesis.

38. The method of paragraph 35, further comprising repeating at least once the step of fastening the prosthesis to the bone with a bone screw, such that the prosthesis is attached to the bone with at least two bone screws.

39. The method of paragraph 38, wherein at least one of the bone screws is introduced first through an aperture in the prosthesis and then into the bone, and wherein at least another of the bone screws is introduced first through the bone and then into an aperture in the prosthesis.

40. The method of paragraph 35, wherein the step of fastening the prosthesis to the bone with a bone screw includes threading the bone screw into an aperture in the prosthesis.

41. The method of paragraph 35, wherein the bone screws are positioned such that all lie within the surface of the body and bone.

42. The method of paragraph 35, further comprising drilling a guide channel through the bone for receiving the bone screw, prior to the step of fastening the prosthesis to the bone with a bone screw.

43. The method of paragraph 32, further comprising selecting the prosthesis from a kit comprising a plurality of prostheses configured for different articulating surfaces.

44. The method of paragraph 32, further comprising selecting the prosthesis from a kit comprising a plurality of prostheses configured for different sizes of the same articulating surface.

45. The method of paragraph 32, the prosthesis including at least one projection that extends from the body, further comprising inserting the projection into the bone to limit movement of the prosthesis.

46. The method of paragraph 32, further comprising repeating the steps of cutting and attaching to repair an articulating surface on the opposing skeletal member.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A prosthetic device for partial replacement of an articulating surface of a movable joint, the movable joint including a bone defining the articulating surface and an opposing skeletal member that contacts the articulating surface, the device comprising:
a body configured to be connected to the bone to replace one of two contiguous regions of the articulating surface and also to replace subchondral bone material underlying the one region, the body including a replacement region so that a hybrid articulation surface is formed with the replacement region and the other contiguous region for movable contact with the opposing skeletal member.

2. The prosthetic device of claim 1, wherein the movable joint is an elbow.

3. The prosthetic device of claim 2, wherein the bone is a radius.

4. The prosthetic device of claim 3, wherein the replacement region is configured to be located on the proximal radius and to articulate with at least one of the radial notch of the ulna and the capitulum of the distal humerus.

5. The prosthetic device of claim 2, wherein replacement region corresponds to at least a portion of the coronoid process of the ulna and is configured to articulate with at least one of the radial head and the trochlea of the distal humerus.

6. The prosthetic device of claim 5, wherein the replacement region is configured to replace at least a portion of the radial notch of the ulna.

7. The prosthetic device of claim 1, wherein the articulating surface has a perimeter, and wherein the one contiguous region extends to the perimeter.

8. The prosthetic device of claim 1, wherein the body defines at least one aperture configured to receive fasteners to connect the body to the bone.

9. The prosthetic device of claim 8, wherein the at least one aperture includes an aperture configured to receive a bone screw threadably that is introduced first through the bone and then into the aperture.

10. The prosthetic device of claim 8, wherein the at least one aperture includes an aperture configured to receive a bone screw that is introduced first through the aperture and then into bone.

11. The prosthetic device of claim 1, further comprising at least one projection that extends from the body, the projection being configured to be received in the bone to limit movement of the body.

12. The prosthetic device of claim 1, wherein the body defines an inner surface configured to abut one or more cut bone surfaces.

13. The prosthetic device of claim 12, wherein the inner surface is configured to promote bone adhesion.

14. The prosthetic device of claim 12, wherein at least a substantial portion of the inner surface is formed by one or more generally planar regions.

15. The prosthetic device of claim 14, wherein the inner surface includes a pair of regions configured to abut a pair of cut bone surfaces extending transversely relative to one another.

16. The prosthetic device of claim 1, wherein the body is formed at least substantially of a non-bone material.

17. A prosthetic device for partial replacement of an articulating surface on a bone that articulates with an opposing skeletal member in a movable joint, comprising:
a body configured to be connected to the bone to replace one of two surface regions of the articulating surface for movable contact with the opposing skeletal member and also to replace subchondral bone material underlying the one surface region, the two surface regions alternately contacting a surface site on the opposing skeletal member as the bone moves relative to the opposing skeletal member.

18. The prosthetic device of claim 17, wherein the body includes a replacement surface corresponding to the one surface region so that a hybrid articulation region is formed with the replacement surface and the other surface region for contact with the opposing skeletal member.

19. The prosthetic device of claim 17, wherein the articulating surface has a perimeter, and wherein the one surface region extends to the perimeter.

20. The prosthetic device of claim 17, wherein the articulating surface is provided by one of (1) the radial head of a radius, and (2) the coronoid process of an ulna.

21. The prosthetic device of claim 17, wherein the body defines an inner surface configured to abut the bone at one or more cut bone surfaces, and wherein at least a substantial portion of the inner surface is formed by one or more generally planar regions.

22. A prosthetic device for partial replacement of an articulating surface on an end segment of a bone, the articulating surface being at least substantially continuous and having a perimeter, the device comprising:
a body configured to be connected to the bone to replace subchondral bone material and including a replacement surface for a missing or removed portion of the articulating surface adjacent the subchondral bone material, the replacement surface including part of the perimeter so that a hybrid surface is formed for movable contact with an opposing skeletal member.

23. A prosthetic device for partial replacement of an articulating surface of an elbow joint, comprising:
a body configured to be connected to a bone of the elbow joint to replace one of two adjacent surface regions of an articulating surface of the bone and also to replace subchondral bone material underlying the one surface region, thereby providing movable contact with one or more opposing skeletal members of the elbow joint.

24. A method of repairing an articulating surface of a bone, the articulating surface contacting an opposing skeletal member in a movable joint, comprising:
removing one of two contiguous regions of the articulating surface and subchondral bone material underlying the one region, to create a cut surface of the bone; and
attaching a prosthesis to the bone adjacent the cut surface so that the one region of the articulating surface is replaced with a prosthetic region having an outer surface configured to contact the opposing skeletal member movably.

25. The method of claim 24, wherein the step of removing includes a step of creating one or more substantially planar surfaces on the bone, and wherein the step of attaching a prosthesis includes a step of abutting the one or more substantially planar surfaces with an inner surface of the prosthesis.

26. The method of claim 25, wherein the step of creating creates at least two substantially planar surfaces extending transversely to one another.

27. The method of claim 24, wherein the step of attaching a prosthesis includes a step of fastening the prosthesis to the bone with a plurality of bone screws.

28. The method of claim 27, wherein the step of fastening the prosthesis includes a step of introducing a bone screw first through the bone and then into threaded engagement with an aperture in the prosthesis.

29. The method of claim 24, the prosthesis including at least one projection that extends from the body, further comprising a step of inserting the projection into the bone to limit movement of the prosthesis.

30. The method of claim 24, further comprising repeating the steps of removing and attaching to repair an articulating surface on the opposing skeletal member.
